(19) United States
(12) Patent Application Publication
(10) Pub. No.: US 2026/0165969 A1
(43) Pub. Date: Jun. 18, 2026

(54) METHOD, DEVICE, AND KIT FOR PRODUCING A PHARMACEUTICALLY ACTIVE COATING

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/407,823

(22) Filed: Dec. 3, 2025

(30) Foreign Application Priority Data
Dec. 12, 2024 (EP) 24219291.2

(51) Int. Cl. A61K 9/16; A61L 27/34; A61L 27/54; A61P 31/04

(52) U.S. Cl. CPC A61K 9/1676; A61K 9/1647; A61L 27/34; A61L 27/54; A61P 31/04; A61L 2300/406

(57) ABSTRACT

A method for coating implants with a polymer layer containing at least one pharmaceutical active ingredient. A kit-of-parts suitable for carrying out the method, and to a corresponding device obtained from the kit-of-parts.

### CROSS-REFERENCE TO RELATED APPLICATION

This application claims priority pursuant to 35 U.S.C. 119 (a) to European Application No. 24219291.2, filed Dec. 12, 2024, which application is incorporated herein by reference in its entirety.

### FIELD

The present invention relates to a method for coating implants with a polymer layer containing at least one pharmaceutical active ingredient. The invention also relates to a kit-of-parts suitable for carrying out the method according to the invention, and to a corresponding device obtained from the kit-of-parts.

### BACKGROUND OF THE INVENTION AND PRIOR ART

The present invention relates to a method for coating implants with a polymer layer containing at least one pharmaceutical active ingredient. The method is particularly suitable for the intraoperative coating, i.e., coating at a time close to an operation, of implants commonly used in trauma surgery and orthopedics, such as intramedullary nails and osteosynthesis plates, with polymers in which pharmaceutical active ingredients, preferably anti-infectives, are dispersed. The coated implants are intended to be used particularly in septic revisions in trauma surgery and orthopedics. After they have been implanted into the human body, the coated implants act as local drug release systems. Aqueous body fluids, such as blood and wound secretions, can dissolve the pharmaceutical active ingredients from the polymer layer and release them into the surroundings of the implants. The surfaces of the implants can be temporarily protected from microbial colonization, for example, by the pharmaceutical active ingredients released.

Intramedullary nails and osteosynthesis plates are prior art and are used successfully all over the world for the treatment of bone fractures in trauma surgery. Unfortunately, after the fractures have been treated, infections of the surrounding bone and soft tissue with microorganisms, in particular with bacteria, and commonly with skin germs such as Staphylococcus aureus and Staphylococcus epidermidis, can sometimes arise. When such infections occur, surgical remediation of the infected tissue areas is absolutely necessary, during which remediation areas of infected tissue are removed. Furthermore, systemic antibiotics are commonly used after debridement to reduce germs. It would be desirable for mechanical stabilization of the fractured bone tissue to still be possible after surgical remediation, e.g., using intramedullary nails or osteosynthesis plates, and for these to simultaneously be able to release local anti-infectives in order to locally suppress microbial germs remaining in the debrided tissue.

Intramedullary nails having an antibiotic-containing polymethyl methacrylate bone cement coating have been known for a long time and show clinical results (N. Walter, M. Rupp, J. Krueckel, Volker Alt: Individual and commercially available antimicrobial coatings for intramedullary nails for the treatment of infected long bone non-unions: a systematic review. Injury 53S3 (2022), pages 75-80). After implantation of the coated intramedullary nails, the antibiotic particles contained in the polymethyl methacrylate bone cement layer are dissolved from the bone cement layer by aqueous body fluids such as wound secretions and blood, leading to a locally high antibiotic concentration at the surface of the coated intramedullary nails. These high local antibiotic concentrations, in combination with systemic antibiotics, reduce the microbial germs remaining after debridement.

Of particular interest are intraoperatively coated intramedullary nails. Intraoperative coating makes it possible to incorporate anti-infectives that are precisely tailored to the germs present into the coating—for example, into the polymethyl methacrylate bone cement. To date, intraoperative coating has been carried out by mixing anti-infectives into the cement powder, mixing the cement powder modified in this way with a monomer solution, and manually shaping the resulting cement paste around the nail to be coated. It is difficult to achieve a uniform layer thickness over the entire length of the intramedullary nail. It may therefore be possible for at least some of the coated nail to have too large a cross-section and to no longer be implantable into the previously debrided bone.

Our own practical experience has shown that relatively small amounts of anti-infectives can be added to the polymethyl methacrylate bone cement because, at higher contents of anti-infectives, the cement paste becomes too viscous to be used to coat intramedullary nails. Furthermore, the adhesion of the cement to metal surfaces is impaired as the levels of anti-infectives increase.

It would be desirable if a coating having a low layer thickness and at the same time a high proportion of anti-infectives were possible. This would eliminate problems with the radial expansion of the intramedullary nails and at the same time enable high local release of anti-infectives.

In addition to coating with polymethyl methacrylate bone cement containing anti-infectives, direct coating of implants with antibiotics and with antibiotics suspended or dissolved in polymers is also possible.

US 2007/0134287 A describes an antibiotic coating solution that consists of a readily evaporating solvent and an antibiotic dissolved therein.

U.S. Pat. No. 11,517,650 B describes a similar coating solution, in which antibiotics are dissolved in a rapidly evaporating solvent, and energy is supplied by irradiating ultrasound in order to accelerate the dissolution process. The solution is then used for antibiotic coating.

EP 1 243 259 B discloses a coating consisting of polymethyl methacrylate and a hydrophilic polymer, such as a polyether, in which an antibiotic is suspended. The disadvantage of the disclosed copolymers is that the polymer coating obtained according to EP 1 243 259 B is relatively thick. In addition, it is necessary to add the hydrophilic polyether to the coating in order to achieve sufficient release of the antibiotic. The hydrophilic polyether is water-soluble and acts as a pore-forming agent.

A similar concept is described in EP 1 112 095 B. This document proposes a layer of D,L-polylactide in which an antibiotic is suspended. The coating is carried out by first dissolving the polylactide in a rapidly evaporating solvent and then suspending antibiotic particles in this solution. This suspension is applied to implant surfaces, the solvent evaporates, and a D,L-polylactide film remains. The antibiotic particles are fixed in the D,L-polylactide film and can be dissolved out of said film by the action of aqueous body fluids, such as wound secretions and blood. The D,L-polylactide film can be hydrolyzed by the action of water or aqueous solutions and decomposes to yield water-soluble secondary products.

The coating methods described in the prior art are generally complex to implement and time-consuming. Furthermore, the methods of the prior art only allow for limited variation in the pharmaceutical active ingredients, and selecting suitable pharmaceutical active ingredients immediately before or during an operation is not possible. In addition, the methods described in the prior art are disadvantageous in that they usually result in relatively thick coatings, and unsuitable components have to be used to enable release from the polymer matrix.

### OBJECT OF THE INVENTION

The object of the present invention is to develop a simple, time-saving method for coating articles with a pharmaceutically active polymer layer containing at least one pharmaceutical active ingredient. The coating method shall be designed such that surgical staff can easily incorporate any pharmaceutical active ingredients into the coating. The polymer layer shall be made of a biocompatible polymer that is not hydrolytically or enzymatically degraded in the human body. Furthermore, it shall be possible to apply the polymer layer to surfaces such as implants and osteosynthesis plates using the simplest means under surgical conditions. The polymer layer shall not detach from the surface in the presence of water at 37° C. within 7 days. Any health risks to surgical staff from toxic solvents during the coating process shall be avoided. Furthermore, the method shall make it possible to produce polymer layers having a minimum proportion of active ingredients of 10 wt. %. Furthermore, the polymer layer shall not soften due to the effects of aqueous body fluids and shall have a low but constant layer thickness. Finally, the polymer coating to be produced according to the invention shall have good water and temperature resistance.

For the application of the polymer layer, a kit-of-parts shall be provided, having components suitable for the production and application of a coating solution containing active ingredients.

Another object is to provide a device obtained from the kit-of-parts.

The objects of the present invention are achieved by the measures according to claims 1 to 15.

### SUMMARY OF THE INVENTION

The present objects are, firstly, achieved by a method for producing a pharmaceutically active polymer coating.

The method according to the invention is then characterized by at least the following method steps:
a) dissolving a non-hydrolyzable polymer or copolymer which is characterized by a number-average molar mass of greater than 200,000 g/mol, more preferably a number-average molar mass of greater than 400,000 g/mol, more preferably a number-average molar mass of greater than 600,000 g/mol, determined by gel permeation chromatography, in a solvent or solvent mixture which is characterized by a vapor pressure of at least 95 hPa at 20° C. and standard pressure, forming a polymer solution 1;
b) dissolving or suspending at least one pharmaceutical active ingredient in the polymer solution 1, forming a polymer solution 2;
c) applying the polymer solution 2 to the surface of an article to be coated; and
d) evaporating the solvent or solvent mixture such that a polymer layer remains on the surface of the article to be coated, in which polymer layer the at least one pharmaceutical active ingredient is dispersed,
characterized in that the polymer layer does not comprise any pore-forming agents which are soluble in aqueous solutions.

In addition, the present invention also relates to a kit-of-parts for carrying out the method according to the invention, comprising the following components:
e) a solvent-resistant container having an opening;
f) a solvent-resistant closure element suitable for closing the container;
g) an application aid;
h) a polymer solution 1 as defined in the method according to the invention;
i) optionally, one or more mixing bodies; and
j) optionally, one or more pharmaceutical active ingredients in separate packaging.

The scope of protection of the present invention also covers a device for carrying out the method according to the invention, which device is obtained by means of the medical kit-of-parts or can be provided independently thereof.

Furthermore, the scope of the present invention also covers a medical implant which is provided with a polymer coating obtainable by the method according to the invention. The use of the medical implant for the prevention of infections after a surgical procedure is also claimed here.

The present invention will be described in detail below.

### DETAILED DESCRIPTION OF THE INVENTION

### i. Definitions

In the sense of the present invention, the term polymer solution 1 denotes at least one polymer or copolymer in which the polymer or copolymer has a number-average molar mass of greater than 200,000 g/mol, more preferably a number-average molar mass of greater than 400,000 g/mol, more preferably a number-average molar mass of greater than 600,000 g/mol, determined by gel permeation chromatography, and is present in a solvent or solvent mixture having a vapor pressure of at least 95 hPa at 20° C. and standard pressure, i.e., at 1,013.25 hPa.

In the sense of the present invention, the term polymer solution 2 denotes a solution and/or suspension in which a pharmaceutical active ingredient is dissolved or suspended in the polymer solution 1.

In the sense of the present invention, the term application aid denotes any element which is suitable for distributing, preferably uniformly, a solution or suspension over a surface. In particular, it includes painting elements, spraying elements, or even basins for immersion.

In the sense of the present invention, the term mixing body denotes an element which is suitable for thoroughly mixing, in particular mechanically, a solution or suspension. The term mixing body includes in particular mixing rods such as stirrer rods, but also mixing elements such as balls or magnetic stirrers.

In the sense of the present invention, where reference is made to explicitly designated numerical values in a range from X to Y or from at least X to at least Y or from greater than X to greater than Y, etc., this also includes, in particular, all the values implicitly lying therebetween that are suggested by the specification of the decimal places. Thus, if a value is between 1 and 10, it also includes 2, 3, 4, 5, 6, 7, 8, and 9. If a value is between 1.0 and 2.0, it also includes 1.1, 1.2, 1.3, 1.4, 1.5, 1.6, 1.7, 1.8, and 1.9. If a value is between 1.00 and 1.10, it also includes 1.01, 1.02, 1.03, 1.04, 1.05, 1.06, 1.07, 1.08, and 1.09.

### ii. Method

The present invention firstly relates to a method for coating surfaces of articles, e.g., implants, with a polymer layer in which at least one pharmaceutical active ingredient is dispersed. The method according to the invention can be carried out, for example, by medical personnel with little effort, yielding an article which is provided with a polymer layer in which a pharmaceutical active ingredient is dispersed. Then, in a suitable location, e.g., in vivo, the article can then release the dispersed pharmaceutical active ingredient over a specific period of time. The method according to the invention comprises the following method steps:
b) dissolving a non-hydrolyzable polymer or copolymer which is characterized by a number-average molar mass of greater than 200,000 g/mol, more preferably a number-average molar mass of greater than 400,000 g/mol, more preferably a number-average molar mass of greater than 600,000 g/mol, determined by gel permeation chromatography, in a solvent or solvent mixture which is characterized by a vapor pressure of at least 95 hPa at 20° C. and standard pressure, forming a polymer solution 1;
c) dissolving or suspending at least one pharmaceutical active ingredient in the polymer solution 1, forming a polymer solution 2;
d) applying the polymer solution 2 to the surface of an article to be coated;
e) evaporating the solvent or solvent mixture such that a polymer layer remains on the article to be coated, in which polymer layer the at least one pharmaceutical active ingredient is dispersed,
characterized in that the polymer layer does not comprise any pore-forming agents.

In the sense of the present invention, a non-hydrolyzable polymer or copolymer is selected which is characterized by a number-average molar mass of greater than 200,000 g/mol, more preferably a number-average molar mass of greater than 400,000 g/mol, more preferably a number-average molar mass of greater than 600,000 g/mol, determined by gel permeation chromatography, and which is preferably suitable for coming into contact with a living organism without having a damaging effect upon said organism.

Furthermore, it is provided according to the invention that the non-hydrolyzable polymer or copolymer be dissolved or suspended in a solvent or solvent mixture which is characterized by a vapor pressure of at least 95 hPa at 20° C. and standard pressure. In the sense of the present invention, the solvent or solvent mixture preferably has the property of evaporating relatively rapidly under normal ambient conditions, such as 20° C. and standard pressure. This is advantageous, so that a polymer dissolved in the solvent or solvent mixture relatively quickly remains as a polymer coating on the surface of the article by applying the polymer solution without additional effort, such as drying in an oven. In the sense of the present invention, a further preferred property of the solvent or solvent mixture is that it can come into contact with a living organism without having any damaging effects, such as a toxic effect, upon said organism.

It is therefore particularly provided according to the invention that, firstly, a polymer solution 1 be formed from a polymer or copolymer having a number-average molar mass of greater than 200,000 g/mol, more preferably a number-average molar mass of greater than 400,000 g/mol, more preferably a number-average molar mass of greater than 600,000 g/mol, determined by gel permeation chromatography, in a suitable solvent or solvent mixture which is characterized by a vapor pressure of at least 95 hPa at 20° C. and standard pressure, and none of said substances in the polymer solution 1 have any damaging effects, such as a toxic effect, upon living organisms.

Suitable non-hydrolyzable polymers or copolymers for use in the method according to the invention have specific preferred number-average molar masses (Mn). Thus, in the sense of the present invention, preferred polymers or copolymers have a number-average molar mass (Mn) of greater than 200,000 g/mol—for example, a number-average molar mass of greater than 200,000 g/mol, 210,000 g/mol, 220,000 g/mol, 230,000 g/mol, 240,000 g/mol, 250,000 g/mol, 260,000 g/mol, 270,000 g/mol, 280,000 g/mol, 290,000 g/mol, or greater than 300,000 g/mol, 310,000 g/mol, 320,000 g/mol, 330,000 g/mol, 340,000 g/mol, 350,000 g/mol, 360,000 g/mol, 370,000 g/mol, 380,000 g/mol, 390,000 g/mol, or greater than 400,000 g/mol, 410,000 g/mol, 420,000 g/mol, 430,000 g/mol, 440,000 g/mol, 450,000 g/mol, 460,000 g/mol, 470,000 g/mol, 480,000 g/mol, 490,000 g/mol, or greater than 500,000 g/mol, 510,000 g/mol, 520,000 g/mol, 530,000 g/mol, 540,000 g/mol, 550,000 g/mol, 560,000 g/mol, 570,000 g/mol, 580,000 g/mol, 590,000 g/mol, or greater than 600,000 g/mol.

The number-average molar mass of the non-hydrolyzable polymers and copolymers to be used according to the invention is particularly preferably greater than 550,000 g/mol.

In the sense of the invention, the number-average molar mass (Mn) of the polymers or copolymers is determined by gel permeation chromatography (GPC).

According to the invention, it has been found that non-hydrolyzable polymers and copolymers having number-average molar masses of greater than 200,000 g/mol result in polymer layers that are not too sticky and can be readily applied to a surface. According to the invention, it has also been found that polymers and copolymers which are characterized by the number-average molar masses preferred according to the invention are outstandingly suitable for producing polymer layers which adhere to surfaces, are not brittle or cracked, and can absorb and release pharmaceutical active ingredients.

According to the invention, preference is given to using non-hydrolyzable polymers or copolymers for the polymer solution 1 which have a glass transition temperature (Tg) of greater than 47° C. Preferably, the polymer or copolymer for the polymer solution 1 has a glass transition temperature of greater than 60° C., more preferably greater than 80° C., even more preferably greater than 100° C. The preferred glass transition temperature (Tg) for polymers or copolymers used according to the invention in the polymer solution 1 is therefore greater than 47° C., 48° C., 49° C., or greater than 50° C., 51° C., 52° C., 53° C., 54° C., 55° C., 56° C., 57° C., 58° C., 59° C., or greater than 60° C., 61° C., 62° C., 63° C., 64° C., 65° C., 66° C., 67° C., 68° C., 69° C., or greater than 70° C., 71° C., 72° C., 73° C., 74° C., 75° C., 76° C., 77° C., 78° C., 79° C., or greater than 80° C., 81° C., 82° C., 83° C., 84° C., 85° C., 86° C., 87° C., 88° C., 89° C., or greater than 90° C., 91° C., 92° C., 93° C., 94° C., 95° C., 96° C., 97° C., 98° C., 99° C., or greater than 100° C.

According to the invention, it was surprisingly found that non-hydrolyzable polymers or copolymers having glass transition temperatures (Tg) of greater than 47° C., preferably greater than 60° C., more preferably greater than 80° C., in particular greater than 100° C., are suitable in the sense of the present invention, since these polymers or copolymers remained stable for a period of at least 7 days at a temperature of 37° C. in aqueous medium without dissolving. Such water and temperature resistance is desirable when an article coated by means of the method according to the invention is placed in vivo at a suitable site of action with the at least one pharmaceutical active ingredient dispersed in the polymer layer.

In the sense of the invention, the glass transition temperature (Tg) is determined by differential scanning calorimetry (DSC) at a heating rate of 10 K/min according to ISO 11357 or DIN 53765.

It is further preferred according to the invention that non-hydrolyzable polymers or copolymers which are a component of the polymer solution 1 according to the invention contain no reactive double bonds. Reactive double bonds refer to those sites in a polymer backbone which have a C—C double bond and which can be brought to an undesirable and uncontrolled reaction by external influences, which arise frequently. Such double bonds can easily oxidize, for example, when exposed to gamma radiation in the presence of oxygen, and it is possible for the double bonds to lead to crosslinking between the polymer chains. This damage can cause the polymer solutions to gel, meaning that the polymer solutions are no longer flowable and therefore can no longer be applied uniformly. Conversely, the polymer solutions according to the invention can be easily sterilized by gamma irradiation if no double bonds are contained in the dissolved polymers, which constitutes an advantage according to the invention for medical applications.

A further preferred property according to the invention of the non-hydrolyzable polymers or copolymers, which are components of the polymer solution 1 in the method according to the invention, is that the polymer layer produced therefrom is not too stiff or too brittle; i.e., it remains flexible and does not crumble even if the coated article is deformed.

In the sense of the present invention, it has been found that, in particular, hydrophobic polymers and copolymers having the properties according to the invention are suitable for carrying out the method according to the invention. According to the invention, it has surprisingly been found that non-hydrolyzable polymers and copolymers of polymethyl methacrylates are particularly suitable for forming the polymer layers in the sense of the present invention. In particular, non-hydrolyzable polymers and copolymers of polymethyl methacrylates which are characterized by the ranges of glass transition temperatures and, optionally, number-average molar masses preferred according to the invention are suitable for implementing the method according to the invention. Furthermore, preference is given to using non-hydrolyzable polymers and copolymers of polymethyl methacrylates which have no crystalline components, i.e., which are preferably completely amorphous. The strong intermolecular interactions that usually play a role in crystalline compounds would negatively affect the properties of the polymer layer by potentially leading to increased brittleness and crack formation. In contrast, the preferably amorphous, more preferably non-hydrolyzable, polymers and copolymers in the sense of the invention form weaker, dispersive interactions, for instance dipole interactions and van der Waals interactions, which, on a macroscopic level, lead to a more shapeable substance without brittleness and cracks. As a result, the polymers and copolymers having the properties according to the invention are particularly suitable for the method according to the invention for forming a polymer layer.

Non-hydrolyzable polymers and copolymers which are derived from polymethyl methacrylates and are characterized by the properties preferred according to the invention are preferred in the sense of the method according to the invention, because they have excellent biocompatibility. They can therefore be used in vivo, which is preferred, since the polymer layers formed using the method according to the invention are preferably used for in vivo applications.

The polymers and copolymers derived from polymethyl methacrylates are non-cytotoxic and resistant to hydrolysis.

When reference is made to copolymers of polymethyl methacrylate in the sense of the present invention, this means in particular copolymers which can be produced by polymerization from methyl methacrylate and the comonomers methyl acrylate, ethyl acrylate, ethyl methacrylate, and styrene.

In the sense of the present invention, it has been found that the use of non-hydrolyzable polymers and copolymers having the properties preferred according to the invention yields polymer coatings which have the desired brittleness, can absorb a high content of pharmaceutical active ingredients, and reliably release the pharmaceutical active ingredient at the target site.

A particular feature according to the invention is that the non-hydrolyzable polymers and copolymers do not require additives which usually have to be added in the prior art, such as pore-forming agents.

In the sense of the present invention, pore-forming agents are in particular components in the polymer layer which can generate pores in the water-insoluble polymer layer under the action of water. Pore-forming agents are also in particular substances that are water-soluble and/or hydrolyzable. Examples of pore-forming agents include poly-α-hydroxy acids or oligomers for producing poly-a-hydroxy acids, in particular polyglycols, polylactides such as D,L-polylactides, polytyrosine carbonates and oligomeric ethylene glycol compounds, in particular oligomeric ethylene glycol compounds having a number-average molar mass in the range from 120 g/mol to 35,000 g/mol, starch, gelatin, cellulose, a-hydroxy acids such as lactic acid, in particular D,L-lactic acid, sugar alcohols such as ethylene glycol and diethylene glycol, mannitol, xylene, and sorbitol, amino acids such as alanine, glycine, tyrosine, and serine, and surfactants such as fatty alcohols.

Pore-forming agents include in particular substances which are substantially completely soluble and/or decompose within 24 hours at a concentration of 1 g/liter in phosphate-buffered saline solution (i.e., 137 mM sodium chloride, 2.7 mM potassium chloride, and 12 mM total phosphate, pH 7.4) at 25° C.

Methods known in the prior art for producing a polymer coating with a pharmaceutical active ingredient dispersed therein require further additives such as pore-forming agents to be added to the polymer coating. This modifies the polymer coating so that it can absorb sufficient quantities of pharmaceutical active ingredients and release them again at the target site. The method according to the invention yields a polymer coating that does not require such additives.

Overall, non-crystalline, hydrophobic polymers or copolymers of polymethyl methacrylates having number-average molar masses (Mn) of greater than 200,000 g/mol, more preferably greater than 400,000 g/mol, more preferably greater than 600,000 g/mol, and glass transition temperatures (Tg) of greater than 47° C., more preferably greater than 60° C., more preferably greater than 80° C., more preferably greater than 100° C., are particularly preferred in the sense of the present invention.

Furthermore, preference is given according to the invention to using a solvent or solvent mixture which contains less than 8 wt. % of water to produce the polymer solution 1 according to the invention. The solvent or solvent mixture more preferably comprises less than 7 wt. %, less than 6 wt. %, less than 5 wt. %, less than 4 wt. %, less than 3 wt. %, less than 2 wt. % water.

Contamination of the solvent or solvent mixture with water can cause the active ingredient particles in the polymer solution 2 to clump together in the case of hygroscopic pharmaceutical active ingredients. Therefore, a water content which is as low as possible, less than 2 wt. %, is particularly preferred.

It is advantageous according to the invention if the solvent or solvent mixture is adapted to the polymer or copolymer for the polymer solution 1; in other words, a solvent or solvent mixture is preferably used to increase the solubility of certain monomers or comonomers for the polymer or copolymer.

In the sense of the method according to the invention, solvents or solvent mixtures preferably include acetic acid esters, acetoacetic acid esters, acetone, diacetone alcohol, ethyl methyl ketone, butyl methyl ketone, and/or mixtures thereof. It is also preferred in the sense of the invention to use mixtures with an acetic acid ester and another solvent, including acetoacetic acid ester, preferably ethyl acetoacetate, acetone, diacetone alcohol, ethyl methyl ketone, and/or butyl methyl ketone. A particularly preferred solvent in the sense of the present invention is an acetic acid ester, in particular ethyl acetate.

Ethyl acetate is non-toxic, has a vapor pressure of 98.4 hPa at 20° C. and standard pressure, and a boiling point of 77° C. at standard pressure. Ethyl acetate therefore evaporates relatively quickly at room temperature. Another advantageous property of ethyl acetate is that its smell is perceived as pleasant by most people. If, therefore, a polymer-coated article is produced in the sense of the method according to the invention, which article may have to be suitable for an in vivo medical application, ethyl acetate is preferred, not only for its excellent physical and toxicological qualities, but also for its olfactory perception, by professionals and, if applicable, patients. Ethyl acetate is also easy to handle and, unlike other solvents with similar properties, does not damage most typical plastic articles used in the kit-of-parts according to the invention described further below.

According to the invention, it has surprisingly been found that solutions of polymers and copolymers proposed according to the invention in ethyl acetate form very well-adhering and stable polymer layers when they are applied to surfaces of articles, preferably metallic surfaces of articles, and the solvent evaporates. The evaporation process essentially takes place within one to two minutes at a room temperature of 23° C. Therefore, ethyl acetate is particularly well-suited in the sense of the invention for producing the coatings relatively quickly and without great effort.

The polymer solution 1, comprising a polymer or copolymer preferred according to the invention and a solvent or solvent mixture proposed according to the invention, preferably has a polymer content between 2 wt. % and 18 wt. %, i.e., for example, at least 2 wt. %, at least 3 wt. %, at least 4 wt. %, at least 5 wt. %, at least 6 wt. %, at least 7 wt. %, at least 8 wt. %, at least 9 wt. %, preferably at least 10 wt. %. According to the invention, a polymer content of less than or equal to 18 wt. %, less than or equal to 17 wt. %, less than or equal to 16 wt. %, less than or equal to 15 wt. %, less than or equal to 14 wt. %, less than or equal to 13 wt. %, less than or equal to 12 wt. %, less than or equal to 11 wt. % is also preferred.

A polymer content of 9 wt. % to 11 wt. % is particularly preferred. At this polymer concentration, the polymer solution can be readily mixed with pharmaceutical active ingredients and then readily applied to surfaces.

Polymer concentrations higher than 18 wt. % are possible, but as the polymer concentration increases, properties such as the spreadability or sprayability of the polymer solution may deteriorate, since the polymer solution may become clumped or viscous as a result.

In contrast, if the polymer concentration is too low, this can have a negative effect upon the polymer layer formation properties of polymer solution 2. This means, for example, that a complete polymer layer is not formed when a polymer solution 2 with too low a polymer concentration is applied, but, rather, a polymer layer with holes.

In the sense of the present invention, at least one pharmaceutical active ingredient is dissolved or dispersed in the polymer solution 1 obtained. According to the invention, in principle, any pharmaceutical active ingredient which can be brought into contact with a polymer solution 1 without decomposing is suitable. According to the invention, it is possible for the pharmaceutical active ingredient to dissolve in the polymer solution 1 or to be suspended in the polymer solution 1.

Preferably, the pharmaceutical active ingredient is suspended in the polymer solution 1. Many currently available pharmaceutical active ingredients have only low solubility in common solvents. This often results in a low concentration of the pharmaceutical active ingredient in a solution of the polymer, which in turn results in a low concentration of the pharmaceutical active ingredient in a coating obtained from a solution. This means that bioavailability in use is low, and it is problematic to transport sufficient quantities of the pharmaceutical active ingredient to the desired site of action. Therefore, suspensions of the pharmaceutical active ingredient in the solvent or solvent mixture are preferred, since this makes it possible to achieve higher concentrations.

In the sense of the present invention, the solution or suspension obtained when a pharmaceutical active ingredient is dissolved or suspended in a polymer solution 1 according to the invention is referred to as polymer solution 2, even if it is a suspension.

In the sense of the present invention, the polymer solution 2, comprising the solvent or solvent mixture, the dissolved polymer or copolymer, and the at least one dissolved or suspended pharmaceutical active ingredient, is then applied to the surface of an article on which a polymer layer having the pharmaceutical active ingredient dispersed therein is to be obtained.

After application, the selected solvent preferably evaporates relatively quickly under standard ambient conditions, preferably without the need for any additional effort such as drying in a suitable oven or drying with a hot air dryer. This leaves an article having a polymer layer in which the pharmaceutical active ingredient is dispersed.

In the sense of the method according to the invention, at least one pharmaceutical active ingredient is dissolved or suspended in the polymer solution 1 in order to obtain a polymer solution 2. Possible pharmaceutical active ingredients include, for example, active ingredients from the classes of anti-infectives, bacteriophages, cytostatics, analgesics, hormones, and growth factors. Articles coated with the polymer solution 2 can then continuously release active ingredients, in particular from the listed classes of active ingredients, locally at a site of action, also in vivo, over a specific period of time.

The term “anti-infectives” refers to antimicrobially active ingredients, which can be of microbial, semi-synthetic, or even synthetic origin.

In the sense of the invention, the term “bacteriophages” refers to active ingredients that are used in a targeted manner antimicrobially for therapeutic purposes.

The term cytostatics refers to active ingredients that have a toxic effect upon cells and are usually used to treat cancers.

The term analgesics refers to active ingredients that have a pain-killing or pain-relieving effect.

Hormones and growth factors are substances produced by the body, or active ingredients derived from substances produced by the body, that can influence the body's own processes, such as metabolism.

In the sense of the method according to the invention, pharmaceutical active ingredients such as gentamicin, tobramycin, amikacin, clindamycin, colistin, moxifloxacin, levofloxacin, ciprofloxacin, vancomycin, teicoplanin, daptomycin, doxycycline, ampicillin, amoxicillin, meropenem, fluconazole, amphotericin B, caspofungin, micafungin, and mixtures of the aforementioned active ingredients are particularly preferably dissolved or suspended in the polymer solution 2.

In the sense of the present method according to the invention, it is very particularly preferred if the pharmaceutical active ingredients are in a particulate form before being dissolved or suspended; in other words, the active ingredients are present as a solid characterized by specific particle size ranges. The pharmaceutical active ingredients, before dissolution or suspension, are preferably present at a particle size of less than 250 μm, preferably less than 100 μm. The particle size is in particular less than 240 μm, less than 230 μm, less than 220 μm, less than 210 μm, less than 200 μm, less than 190 μm, less than 180 μm, less than 170 μm, less than 160 μm, less than 150 μm, less than 140 μm, less than 130 μm, less than 120 μm, less than 110 μm. In the sense of the invention, the particle size is determined by a sieving process (sieve fractionation).

According to the invention, it has been found that particularly finely sieved, particulate pharmaceutical active ingredients are preferable, since, in the form of a suspension, they promote the formation of a uniform polymer coating. Larger particles tend to clump together or form a non-uniform coating, which is not preferred according to the invention. If the aim is to dissolve the pharmaceutical active ingredients, small particles are also preferred, because smaller particles generally dissolve faster than larger ones due to their unfavorable surface-to-volume ratio.

In order to promote the formation of a uniform suspension or complete dissolution of the pharmaceutical active ingredient when carrying out the method according to the invention, it is preferred to assist the dissolution or suspension process using a mixing body. For example, mixing rods, such as standard laboratory glass or stirrer rods, can be used to stir the pharmaceutical active ingredient in the polymer solution 1 until it dissolves or until it is uniformly suspended in the polymer solution 2. It is also conceivable to use mixing elements such as balls made of glass, ceramic, metal, or other materials, or magnetic stirring elements referred to as magnetic stirrers or stirrer rods. In the case of the various balls, use can preferably be made of a closed container comprising the balls, polymer solution 1, and the pharmaceutical active ingredient, which container is then shaken by hand or using a shaking device. The magnetic stirring element makes it possible to stir the mixture of polymer solution 1 and pharmaceutical active ingredient on a magnetic stirrer plate.

The concentration of pharmaceutical active ingredient to be achieved in a polymer coating obtainable by means of the method according to the invention substantially depends physically upon the polymer content in the polymer solution 2. The same ratio of polymer or copolymer to pharmaceutical active ingredient is preferably present in the polymer solution 2 as is to be obtained in the final polymer coating. In the method according to the invention, a weight ratio of polymers or copolymers to pharmaceutical active ingredients of preferably between 1.00:0.05 and 1.00:1.50 is used. This means that the weight ratio of polymer(s) and copolymer(s) to pharmaceutical active ingredient(s) is, for example, 1.00:0.05 to 1.00:1.50, 1.00:0.30 to 1.00:1.30, 1.00:0.60 to 1.00:1.15, or 1.00:0.95 to 1.00:1.05. It is surprising that the method according to the invention makes it possible to produce coatings which have a higher active ingredient content than their polymer content. This makes it possible to achieve high loading densities on the coated surface.

An advantage of the invention is that the polymer coatings according to the invention can have a very high loading density with pharmaceutical active ingredient, but it is nevertheless not necessary to add additives such as pore-forming agents to the polymer coatings in order for the layer to remain stable at the target site and to continuously release the pharmaceutical active ingredient. Thus, it is possible to achieve a surprisingly high loading density with a pharmaceutical active ingredient in the polymer layer without having to use the components commonly used in the prior art. Thus, the method according to the invention makes it possible to obtain polymer layers which have excellent adhesion properties, are not brittle, and contain large quantities of pharmaceutical active ingredients, without this having a negative effect upon the properties of the polymer coating. Therefore, ratios according to the invention of polymer(s) and copolymer(s) to pharmaceutical active ingredient(s) of 1.00 to greater than or equal to 1.00 are preferred.

If a polymer solution 2 according to the invention has been prepared according to the steps described above, it can in principle be applied to all types of articles in the sense of the method according to the invention, in order to produce a polymer layer thereon in which a pharmaceutical active ingredient is dispersed. In the sense of the method according to the invention, it is particularly preferred if the application is carried out using a simple method which requires no great effort, e.g., by spreading the polymer solution 2 onto a surface, by immersing an article to be coated into a container with the polymer solution 2, or by spraying the polymer solution 2 onto a surface.

In the sense of the present method, it is further preferred if, when the polymer solution 2 is applied, the surface of the article to be coated has a temperature in the range from 10° C. to 30° C., i.e., for example, a temperature of 10° C. to 30° C., 10° C. to 28° C., 10° C. to 26° C., 10° C. to 24° C., 10° C. to 22° C., 10° C. to 20° C.

The temperature of the surface of the article to be coated is preferably between 10° C. and 30° C. At temperatures lower than 10° C., the solvent or solvent mixture may require several minutes to evaporate, which is not preferred in the sense of the present invention. At temperatures of greater than 30° C., it has been found according to the invention that the solvent or solvent mixture may evaporate too quickly, and it is difficult to apply uniform layers to the surface by applying the polymer solution 2.

The method according to the invention makes it possible to produce uniform coatings. In the sense of the present invention, uniform coatings mean complete coverage of the surface of the article with the coating resulting from the polymer solution 2. This also means that the polymer coatings preferably have no cracks or visual signs of brittleness and adhere firmly enough to the surface of the article to prevent them from being removed without the application of force or the action of chemical or physical means.

The polymer coatings obtained according to the invention, in which pharmaceutical active ingredients are dispersed, have a layer thickness of greater than 50 μm. In particular, the layer thicknesses are greater than 60 μm, greater than 70 μm, greater than 80 μm, greater than 90 μm, greater than 100 μm, greater than 110 μm, greater than 120 μm, greater than 130 μm, greater than 140 μm, or greater than 150 μm. However, the layer thicknesses can also be up to 500 μm, up to 450 μm, up to 400 μm, up to 350 μm, or up to 300 μm thick. In particular, the coatings obtained on a surface by means of the method according to the invention have no cracks or signs of brittleness and have a uniform thickness.

It has been found according to the invention that such relatively thin coatings, as can be obtained by the method according to the invention, facilitate the release of the pharmaceutical active ingredient, dispersed in the coatings, at the site of action. In contrast to conventional coatings from the prior art, it is not necessary to add a hydrophilic component, such as a polyether, to the polymer or copolymer.

In principle, in the sense of the method according to the invention, all articles, in particular articles for use in medical applications, more particularly medical implants, can be coated with a polymer coating containing one or more pharmaceutical active ingredients.

It is therefore possible using the method according to the invention, for example, to coat a medical implant, e.g., an intramedullary nail or an osteosynthesis plate, with a pharmaceutically active polymer coating. Due to the easy-to-handle components for the polymer solutions 1 and 2, the method can be carried out without great effort and provides coated implants within minutes. These implants can be inserted and continuously release active ingredients directly at the target site.

The method according to the invention, in particular within the framework of the parameters defined in Table 1, can be carried out as follows:
f) dissolving a non-hydrolyzable polymer or copolymer having a number-average molar mass (Mn) A and a glass transition temperature (Tg) B in a solvent C or a solvent mixture composed of more than one solvent C, forming a polymer solution 1;
g) dissolving or suspending at least one pharmaceutical active ingredient in the polymer solution 1, forming a polymer solution 2;
h) applying the polymer solution 2 to the surface of an article; and
i) evaporating the solvent or solvent mixture such that a polymer layer remains on the article to be coated, in which polymer layer the at least one pharmaceutical active ingredient is dispersed,
characterized in that the polymer layer does not comprise any pore-forming agents.

TABLE 1
Selection of preferred number-average molar masses (Mₙ) A; preferred
glass transition temperature ranges (Tg) B; preferred solvents C,
which are used individually or in the form of solvent mixtures.

| Set-up | Mₙ [g/mol] A | Tg [° C.] B | Solvent C |
|---|---|---|---|
| 1 | >200,000 | >47 | Ethyl acetate |
| | | | Ethyl acetoacetate |
| | | | Acetone |
| | | | Diacetone alcohol |
| | | | Ethyl methyl ketone |
| | | | Butyl methyl ketone |
| 2 | >200,000 | >60 | Ethyl acetate |
| | | | Ethyl acetoacetate |
| | | | Acetone |
| | | | Diacetone alcohol |
| | | | Ethyl methyl ketone |
| | | | Butyl methyl ketone |
| 3 | >200,000 | >80 | Ethyl acetate |
| | | | Ethyl acetoacetate |
| | | | Acetone |
| | | | Diacetone alcohol |
| | | | Ethyl methyl ketone |
| | | | Butyl methyl ketone |
| 4 | >200,000 | >100 | Ethyl acetate |
| | | | Ethyl acetoacetate |
| | | | Acetone |
| | | | Diacetone alcohol |
| | | | Ethyl methyl ketone |
| | | | Butyl methyl ketone |
| 5 | >400,000 | >47 | Ethyl acetate |
| | | | Ethyl acetoacetate |
| | | | Acetone |
| | | | Diacetone alcohol |
| | | | Ethyl methyl ketone |
| | | | Butyl methyl ketone |
| 6 | >400,000 | >60 | Ethyl acetate |
| | | | Ethyl acetoacetate |
| | | | Acetone |
| | | | Diacetone alcohol |
| | | | Ethyl methyl ketone |
| | | | Butyl methyl ketone |
| 7 | >400,000 | >80 | Ethyl acetate |
| | | | Ethyl acetoacetate |
| | | | Acetone |
| | | | Diacetone alcohol |
| | | | Ethyl methyl ketone |
| | | | Butyl methyl ketone |
| 8 | >400,000 | >100 | Ethyl acetate |
| | | | Ethyl acetoacetate |
| | | | Acetone |
| | | | Diacetone alcohol |
| | | | Ethyl methyl ketone |
| | | | Butyl methyl ketone |
| 9 | >600,000 | >47 | Ethyl acetate |
| | | | Ethyl acetoacetate |
| | | | Acetone |
| | | | Diacetone alcohol |
| | | | Ethyl methyl ketone |
| | | | Butyl methyl ketone |
| 10 | >600,000 | >60 | Ethyl acetate |
| | | | Ethyl acetoacetate |
| | | | Acetone |
| | | | Diacetone alcohol |
| | | | Ethyl methyl ketone |
| | | | Butyl methyl ketone |
| 11 | >600,000 | >80 | Ethyl acetate |
| | | | Ethyl acetoacetate |
| | | | Acetone |
| | | | Diacetone alcohol |
| | | | Ethyl methyl ketone |
| | | | Butyl methyl ketone |
| 12 | >600,000 | >100 | Ethyl acetate |
| | | | Ethyl acetoacetate |
| | | | Acetone |
| | | | Diacetone alcohol |
| | | | Ethyl methyl ketone |
| | | | Butyl methyl ketone |

A particularly preferred embodiment of the method according to the invention is described below. In this particularly preferred embodiment, the method according to the invention is characterized by the following method steps:
a) dissolving a polymer or copolymer which is characterized by a number-average molar mass of greater than 200,000 g/mol in an acetic acid ester or a mixture of an acetic acid ester and at least one further solvent which are characterized by a vapor pressure of at least 95 hPa at 20° C. and standard pressure, forming a polymer solution 1;
b) dissolving or suspending at least one particulate anti-infective in the polymer solution 1, forming a polymer solution 2;
c) spreading the polymer solution 2 on the surface of a medical implant; and
d) evaporating the solvent or solvent mixture so that a polymer layer remains on the implant surface, in which polymer layer the at least one particulate anti-infective is dispersed,
characterized in that the polymer layer does not comprise any pore-forming agents.

A further particularly preferred embodiment of the method according to the invention comprises, in particular, the following method steps:
e) dissolving a polymethyl methacrylate or a polymethyl-co-methyl acrylate which is characterized by a number-average molar mass of greater than 200,000 g/mol in an acetic acid ester or a mixture of an acetic acid ester and at least one further solvent which are characterized by a vapor pressure of at least 95 hPa at 20° C. and standard pressure, forming a polymer solution 1;
f) dissolving or suspending at least one particulate anti-infective in the polymer solution 1, forming a polymer solution 2;
g) spreading the polymer solution 2 on the surface of a medical implant;
h) evaporating the solvent or solvent mixture so that a polymer layer remains on the implant surface, in which polymer layer the at least one particulate anti-infective is dispersed,
characterized in that the polymer layer does not comprise any pore-forming agents.

A further particularly preferred embodiment of the method according to the invention comprises, in particular, the following method steps:
i) dissolving a polymethyl methacrylate or a polymethyl-co-methyl acrylate which is characterized by a number-average molar mass of greater than 200,000 g/mol in ethyl acetate or a mixture of ethyl acetate and at least one further solvent which are characterized by a vapor pressure of at least 95 hPa at 20° C. and standard pressure, forming a polymer solution 1;
j) dissolving or suspending at least one particulate anti-infective selected from the group consisting of gentamicin sulfate, vancomycin hydrochloride, clindamycin hydrochloride, daptomycin, and mixtures thereof in the polymer solution 1, forming a polymer solution 2;
k) spreading the polymer solution 2 on the surface of a medical implant;
l) evaporating the solvent or solvent mixture so that a polymer layer remains on the implant surface, in which polymer layer the at least one particulate anti-infective is dispersed,
characterized in that the polymer layer does not comprise any pore-forming agents.

A further particularly preferred embodiment of the method according to the invention comprises, in particular, the following method steps:
m) dissolving 9 wt. % to 11 wt. % of a polymethyl methacrylate or a polymethyl-co-methyl acrylate which is characterized by a number-average molar mass of greater than 200,000 g/mol and a glass transition temperature of greater than 47° C. in ethyl acetate or a mixture of ethyl acetate and at least one further solvent which are characterized by a vapor pressure of at least 95 hPa at 20° C. and standard pressure, forming a polymer solution 1;
n) suspending at least one particulate anti-infective selected from the group consisting of gentamicin sulfate, vancomycin hydrochloride, clindamycin hydrochloride, daptomycin, and mixtures thereof in the polymer solution 1, forming a polymer solution 2;
o) spreading the polymer solution 2 on the surface of a medical implant;
p) evaporating the solvent or solvent mixture so that a polymer layer remains on the implant surface, in which polymer layer the at least one particulate anti-infective is dispersed, characterized in that the polymer layer does not comprise any pore-forming agents.

The method according to the invention leads to coated articles having a polymer layer in which a pharmaceutical active ingredient is dispersed. The article can then be introduced into a living organism, e.g., in the form of an implant, in which the active ingredient is to be released continuously.

The present invention also comprises a kit-of-parts and a device obtained from the kit-of-parts for carrying out the method according to the invention. The kit-of-parts according to the invention and the devices are described in more detail below.

### III. Kit-of-Parts and Device

The present invention also claims a kit-of-parts (hereafter referred to as a kit) for carrying out the method according to the invention. The kit in the sense of the present invention provides all components required by specialist personnel to carry out the method according to the invention.

In addition, however, a device is also claimed which serves to carry out the method according to the invention and is obtained from the kit according to the invention.

The kit according to the invention comprises the following components:
q) a solvent-resistant container having an opening;
ii) a solvent-resistant closure element suitable for closing the container;
iii) an application aid;
iv) a polymer solution 1 as described previously;
v) optionally, a mixing body; and
vi) optionally, one or more pharmaceutical active ingredients in separate packaging.

In the sense of the present invention, therefore, at least one solvent-resistant container, which is suitable for holding solvents or solvent mixtures as described above without being damaged thereby, is provided for the kit. Furthermore, at least one closure element is provided for the container, e.g., a screw or plug closure, which closure element is also solvent-resistant. This makes it possible to provide a polymer solution, optionally prepared beforehand, in the appropriate container for specialist personnel as required.

In the sense of the present invention, the kit is also equipped with an application aid, for which any conceivable element is suitable for uniformly distributing a solution or suspension provided according to the invention on a surface.

In the sense of the present invention, it is advantageous if a finished polymer solution 1 as described above has already been initially charged in the solvent-resistant container. For example, if an implant is to be coated shortly before it is inserted in vivo, it is easier for the specialist personnel if the required quantities of polymer or copolymer do not have to be weighed out first and dissolved in a suitable solvent.

In a preferred embodiment, the kit according to the invention therefore already comprises a polymer solution 1, as described above, in the solvent-resistant container.

In the sense of the present invention, it is also conceivable for a mixing body as described above to be added to the kit for carrying out the method according to the invention. In principle, a pharmaceutical active ingredient can be sufficiently dissolved or dispersed by shaking the closed container with the polymer solution 1. However, to facilitate the process, a mixing body, such as a mixing rod, can be included in the kit, or mixing bodies, such as mixing elements, can be provided directly in the container for the polymer solution 1.

It is also conceivable to configure the kit according to the invention from the outset such that separately packaged pharmaceutical active ingredients are provided, in order to obtain a polymer solution 2 at a target concentration as easily as possible. However, this is not mandatory, since flexibility in the choice of the pharmaceutical active ingredient or in the choice of the concentration thereof may also be desirable.

The kit according to the invention makes it possible, for example, for medical personnel in the operating room to mix any pharmaceutical active ingredients into the polymer solution 1 within a very short time and to also apply the resulting polymer solution 2 to surfaces, e.g., of implants, using the application aid immediately after mixing.

In the sense of the kit according to the invention, it is preferred if the container has an opening which has a diameter of at least 1 cm, preferably at least 2 cm, particularly preferably at least 3 cm. It is advantageous, when mixing the polymer solution 1 with a suitable pharmaceutical active ingredient, for the opening to have one of the preferred minimum sizes. This makes it possible to access the polymer solution 1 with a mixing body such as a stirrer rod or spatula via the opening in the container and to mix it with a pharmaceutical active ingredient.

The opening in the container is preferably provided at the top of the container.

The application aids according to the invention can include all types of articles that make it possible to apply a solution or suspension to the surface of an article. In the sense of the present invention, preference is given to using application aids which can be easily provided in the kit—for example, a brush or an atomizer cap. According to the invention, these application aids can be combined, for example, with the solvent-resistant closure element.

Mixing bodies in the sense of the device according to the invention include in particular the previously described mixing bodies which are suitable for the method according to the invention. This includes, for example, mixing rods such as stirrer rods or spatulas. Alternatively, it is possible for at least one freely movable mixing body to be arranged in the container, wherein plastic balls, glass balls, and ceramic balls are preferred as mixing bodies. In this variant embodiment, the pharmaceutical active ingredient is added to the polymer solution 1, and the mixture is shaken after the container is closed. The action of the mixing body causes the pharmaceutical active ingredients to become suspended or dissolved in the polymer solution 1.

The diameter of the mixing bodies, in particular of the mixing elements, is preferably at most half the size of the internal diameter of the container. This means that, during mixing, the polymer solution still has enough space alongside the mixing body to be able to flow around the mixing body as it moves in the container.

In a preferred variant embodiment of the kit according to the invention, at least some of the components of the kit are physically connected to one another. This preferably relates to the container and the closure element and/or the closure element and the application aid. For example, the closure element can be attached to the container so that it is not accidentally misplaced when a pharmaceutical active ingredient is to be added to the polymer solution 1. It is also preferred if an application aid, such as a brush, is connected to the inside of the closure element, for example, or if the closure element itself is, for example, an atomizer cap.

A particularly preferred variant of the kit according to the invention therefore comprises in particular the following components:
a solvent-resistant container having an opening;
ii) a solvent-resistant closure element suitable for closing the container and optionally attached to the container;
iii) a brush which is optionally attached within the closure element so that, when the closure element is used to close the container, it projects internally into the container;
iv) a polymer solution 1 as described previously;
v) at least one mixing body in the container or a separate mixing rod; and
vi) optionally, one or more pharmaceutical active ingredients in separate packaging.

Proceeding from this kit according to the invention, a corresponding device can be provided before the method according to the invention is carried out. The above corresponding statements regarding the kit according to the invention also apply to the device, which is also claimed.

### IV. Medical Implant and Prevention of Infections

In the sense of the present invention, it is possible to use the method according to the invention to provide an article, such as a medical implant, with a pharmaceutically active polymer coating, which medical implant can then be used to prevent infections in the place of an uncoated implant.

The present invention therefore also relates to a medical implant having the coating described above.

A medical implant according to the invention is provided with a polymer coating obtainable using the method according to the invention, in which polymer coating a pharmaceutical active ingredient is dispersed. The polymer coating is thus characterized by a ratio of the proportion of polymer or copolymer to the proportion of the pharmaceutical active ingredient of between 1.00:0.05 to 1.00:1.50, as previously described.

The polymer coating of the medical implant comprises a polymer having a glass transition temperature (Tg) of greater than 47° C., preferably greater than 60° C., more preferably greater than 80° C., in particular greater than 100° C., and a number-average molar mass (Mn) of greater than 200,000 g/mol, more preferably greater than 400,000 g/mol, more preferably greater than 600,000 g/mol, as previously described.

The polymer coating of the medical implant preferably has no cracks or visible signs of brittleness and is characterized by a layer thickness of 50 μm to 200 μm, as previously described.

A coated medical implant can have a pharmaceutical effect if a pharmaceutical active ingredient has been dispersed in the polymer coating. According to the invention, it has been found that the dispersed active ingredient is released from the matrix of the polymer coating in a warm and aqueous environment.

After surgery, the risk of infection is usually increased because bone or tissue material has been exposed to the external environment—for example, through incisions. In addition, patients who undergo surgery are often weakened, reducing their body's ability to fight infection.

In the sense of the present invention, a medical implant which has been provided with a pharmaceutically active polymer coating using the method according to the invention can, in particular, be an intramedullary nail or an osteosynthesis plate, which then acts to prevent infection at the implanted site in vivo. Intramedullary nails are used in intramedullary nailing to correct fractures of long bones. An osteosynthesis plate is used in osteosynthesis, which is a surgical procedure for treating bone fractures wherein the bone fragments are joined together with the aid of this plate.

In the sense of the present invention, a medical implant which has been provided with a pharmaceutically active polymer coating using the method according to the invention can therefore prevent infection, in particular after surgery in the event of a bone fracture, such as intramedullary nailing.

### EXAMPLES

### r) Investigations Regarding the Properties of Polymer Coatings

Polymethyl methacrylates and polymethyl methacrylate-co-methyl acrylates having number-average molar masses of ˜100,000 g/mol, ˜200,000 g/mol, ˜550,000 g/mol, ˜700,000 g/mol, and ˜1,250,000 g/mol were dissolved at 5.0 wt. % and 10 wt. % in ethyl acetate. These solutions were painted onto a stainless steel surface, and the properties of the remaining polymer layer were examined after the ethyl acetate had evaporated. It was found that more brittle and more poorly adhering coatings were obtained with polymethyl methacrylate having a number-average molar mass of less than 200,000 g/mol than with polymethyl methacrylate and polymethyl methacrylate-co-methyl acrylate having a number-average molar mass of greater than or equal to 200,000 g/mol.

### b) Investigations of the Active Ingredient Concentration in Polymer Coatings

For the following tests, use was made of a polymethyl methacrylate-methyl acrylate copolymer having a number-average molar mass of ˜550,000 g/mol and a stainless steel cylinder (CoCr28Mo6, ASTM F75 alloy) having a diameter of 10.0 mm and a length of 60.0 mm. Gentamicin sulfate, vancomycin hydrochloride, clindamycin hydrochloride, and daptomycin were mixed with a 10 wt. % solution of the polymethyl methacrylate-methyl acrylate copolymer in ethyl acetate (Tables 2 and 3). The anti-infectives were present as powders having an average particle size of less than 100 μm. The dispersions formed were milky-cloudy.

Mixtures 1 to 10 were applied to the stainless steel cylinders by brushing. Only half of the steel cylinder, with an area of 10.20 cm², was coated. Five stainless steel cylinders were coated for each mixture. The mass of the coating was determined gravimetrically after the ethyl acetate had evaporated. The coated stainless steel cylinders were then stored for 24 hours in distilled water at 37° C. The stainless steel cylinders were then dried, and the mass loss due to the release of the anti-infectives was determined gravimetrically.

TABLE 2
Compositions of mixtures 1 to 7; constant masses
of solvent and copolymer, varying masses of
the pharmaceutical active ingredient.

| Mixture | Ethyl | Polymethyl methacrylate- | Gentamicin |
|---|---|---|---|
| no. | acetate | co-methyl acrylate | sulfate |
| 1 | 10.00 g | 1.00 g | 0.25 g |
| 2 | 10.00 g | 1.00 g | 0.50 g |
| 3 | 10.00 g | 1.00 g | 0.75 g |
| 4 | 10.00 g | 1.00 g | 1.00 g |
| 5 | 10.00 g | 1.00 g | 1.25 g |
| 6 | 10.00 g | 1.00 g | 1.50 g |
| 7 | 10.00 g | 1.00 g | 2.50 g |

TABLE 3
Compositions of mixtures 8 to 10; constant masses of solvent and copolymer,
varying pharmaceutical active ingredients each at constant mass.

| | | Polymethyl | Vancomycin | Clindamycin | |
|---|---|---|---|---|---|
| Mixture no. | Ethyl acetate | methacrylate | hydrochloride | hydrochloride | Daptomycin |
| 8 | 10.00 g | 1.00 g | 1.0 | — | — |
| 9 | 10.00 g | 1.00 g | — | 1.0 g | — |
| 10 | 10.00 g | 1.00 g | — | — | 1.0 g |

Tables 2 and 3 show the composition of the mixture referred to as polymer solution 2 for mixtures 1 to 10. The influence of increasing active ingredient concentration was investigated using mixtures 1 to 7. The influence of substance identity for the same proportion by mass was investigated using mixtures 8 to 10.

TABLE 4
Gravimetric investigations on five samples a to e
from mixture 1; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 1 | [mg] | at 37° C. [mg] | [mg] | [%] |
| a | 0.0084 | 0.0053 | 0.0031 | 37 |
| b | 0.0107 | 0.0075 | 0.0032 | 30 |
| c | 0.0081 | 0.0057 | 0.0024 | 30 |
| d | 0.0078 | 0.0054 | 0.0024 | 31 |
| e | 0.0102 | 0.0066 | 0.0036 | 35 |
| | | | Average | 33 |
| | | | mass loss | |

The theoretical mass loss upon complete dissolution of the gentamicin sulfate is 20 wt. %. An average mass loss of 33 wt. % was found.

TABLE 5
Gravimetric investigations on five samples a to e
from mixture 2; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 2 | [mg] | at 37° C. [mg] | [mg] | [%] |
| a | 0.0072 | 0.0038 | 0.0034 | 47 |
| b | 0.0065 | 0.0040 | 0.0025 | 38 |
| c | 0.0092 | 0.0052 | 0.0040 | 43 |
| d | 0.0111 | 0.0064 | 0.0047 | 42 |
| e | 0.0070 | 0.0041 | 0.0029 | 41 |
| | | | Average | 42 |
| | | | mass loss | |

The theoretical mass loss upon complete dissolution of the gentamicin sulfate is 33 wt. %. An average mass loss of 42 wt. % was found.

TABLE 6
Gravimetric investigations on five samples a to e
from mixture 3; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 3 | [mg] | at 37° C. [mg] | [mg] | [%] |
| a | 0.0128 | 0.0070 | 0.0058 | 45 |
| b | 0.0113 | 0.0067 | 0.0046 | 41 |
| c | 0.0151 | 0.0092 | 0.0059 | 38 |
| d | 0.0150 | 0.0094 | 0.0056 | 37 |
| e | 0.0144 | 0.0086 | 0.0058 | 40 |
| | | | Average | 40 |
| | | | mass loss | |

The theoretical mass loss upon complete dissolution of the gentamicin sulfate is 43 wt. %. An average mass loss of 40 wt. % was found.

TABLE 7
Gravimetric investigations on five samples a to e
from mixture 4; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 4 | [mg] | at 37° C. [mg] | [mg] | [%] |
| a | 0.0074 | 0.0036 | 0.0038 | 51 |
| b | 0.0075 | 0.0039 | 0.0036 | 48 |
| c | 0.0102 | 0.0052 | 0.0050 | 49 |
| d | 0.0134 | 0.0065 | 0.0069 | 51 |
| e | 0.0152 | 0.0073 | 0.0079 | 52 |
| | | | Average | 50 |
| | | | mass loss | |

The theoretical mass loss upon complete dissolution of the gentamicin sulfate is 50 wt. %. An average mass loss of 50 wt. % was found.

TABLE 8
Gravimetric investigations on five samples a to e
from mixture 5; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 5 | [mg] | at 37° C. [mg] | [mg] | [%] |
| a | 0.0100 | 0.0044 | 0.0056 | 56 |
| b | 0.0104 | 0.0045 | 0.0059 | 57 |
| c | 0.0160 | 0.0066 | 0.0094 | 59 |
| d | 0.0146 | 0.0069 | 0.0077 | 53 |
| e | 0.0098 | 0.0046 | 0.0052 | 53 |
| | | | Average | 56 |
| | | | mass loss | |

The theoretical mass loss upon complete dissolution of the gentamicin sulfate is 55 wt. %. An average mass loss of 56 wt. % was found.

TABLE 9
Gravimetric investigations on five samples a to e
from mixture 6; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 6 | [g] | at 37° C. [g] | [mg] | [%] |
| a | 0.0138 | 0.0052 | 0.0086 | 62 |
| b | 0.0161 | 0.0060 | 0.0101 | 63 |
| c | 0.0152 | 0.0056 | 0.0096 | 63 |
| d | 0.0142 | 0.0053 | 0.0089 | 63 |
| e | 0.0180 | 0.0063 | 0.0117 | 65 |
| | | | Average | 63 |
| | | | mass loss | |

The theoretical mass loss upon complete dissolution of the gentamicin sulfate is 60 wt. %. An average mass loss of 63 wt. % was found.

TABLE 10
Gravimetric investigations on five samples a to e
from mixture 7; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 7 | [mg] | at 37° C. [mg] | [mg] | [%] |
| a | 0.0147 | 0.0043 | 0.0104 | 71 |
| b | 0.0207 | 0.0058 | 0.0149 | 72 |
| c | 0.0180 | 0.0053 | 0.0127 | 71 |
| d | 0.0152 | 0.0043 | 0.0109 | 72 |
| e | 0.0212 | 0.0065 | 0.0147 | 69 |
| | | | Average | 71 |
| | | | mass loss | |

The theoretical mass loss upon complete dissolution of the gentamicin sulfate is 71 wt. %. An average mass loss of 71 wt. % was found.

TABLE 11
Gravimetric investigations on five samples a to e
from mixture 8; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 8 | [mg] | at 37° C. [mg] | [mg] | [%] |
| a | 0.0128 | 0.0065 | 0.0063 | 49 |
| b | 0.0112 | 0.0057 | 0.0055 | 49 |
| c | 0.0120 | 0.0060 | 0.0060 | 50 |
| d | 0.0104 | 0.0052 | 0.0052 | 50 |
| e | 0.0130 | 0.0066 | 0.0064 | 49 |
| | | | Average | 49 |
| | | | mass loss | |

The theoretical mass loss upon complete dissolution of the vancomycin hydrochloride is 50 wt. %. An average mass loss of 49 wt. % was found. The vancomycin hydrochloride was almost completely dissolved out of the coating.

TABLE 12
Gravimetric investigations on five samples a to e
from mixture 9; mass of the polymer coating after
application and after storage in 37° C. warm water.

| | Mass of the | Mass of the coating | Mass | Mass |
|---|---|---|---|---|
| Coating with | coating | after storage in water | loss | loss |
| mixture no. 9 | [mg] | at 37° C. [mg] | [mg] | [%] |
| a | 0.0079 | 0.0044 | 0.0035 | 44 |
| b | 0.0155 | 0.0080 | 0.0075 | 48 |
| c | 0.0118 | 0.0065 | 0.0053 | 45 |
| d | 0.0121 | 0.0066 | 0.0055 | 45 |
| e | 0.0082 | 0.0046 | 0.0036 | 44 |
| | | | Average | 45 |
| | | | mass loss | |

The theoretical mass loss upon complete dissolution of the clindamycin hydrochloride is 50 wt. %. A mass loss of 45 wt. % was found. The clindamycin hydrochloride was almost completely dissolved out of the coating.

TABLE 13
Gravimetric investigations on five samples a to e from
mixture 10; mass of the polymer coating after application
and after storage in 37° C. warm water.

| Coating with | Mass of the | Mass of the coating | Mass | Mass |
|---|---|---|---|---|
| mixture no. | coating | after storage in water | loss | loss |
| 10 | [mg] | at 37° C. [mg] | [mg] | [%] |
| a | 0.0121 | 0.0071 | 0.0050 | 41 |
| b | 0.0134 | 0.0078 | 0.0056 | 42 |
| c | 0.0050 | 0.0031 | 0.0019 | 38 |
| d | 0.0091 | 0.0054 | 0.0037 | 40 |
| e | 0.0106 | 0.0063 | 0.0043 | 40 |
| | | | Average | 40 |
| | | | mass loss | |

The theoretical mass loss upon complete dissolution of the daptomycin is 50 wt. %. An average mass loss of 40 wt. % was found. Residual daptomycin remained in the coating.

It was investigated why higher mean mass losses were achieved for the results presented in Tables 4, 5, 8, and 9 than had been calculated theoretically. To this end, one coating each was examined before storage in warm water and after storage, using a scanning electron microscope (FIG. 1).

It was found that encapsulations or inclusions spread throughout the polymer coating had formed, which then opened when stored in water at 37° C. It is assumed that small particles detach from the polymer layer or else that weighing errors account for the mass loss.

The results show that, within the limits of weighing errors, the antibiotics were virtually quantitatively released from the coatings by the action of water.

### c) Antimicrobial Activity of Coatings 4, 6, 8, and 10

The eluates of tests 4, 6, 8, and 10 were examined for their antimicrobial activity. To this end, inhibition zone tests were carried out using the test germ Bacillus subtilis ATCC 6633 and nutrient agar 1. Heated nutrient agar I was mixed with a spore suspension of Bacillus subtilis, and the inoculated nutrient agar I was poured into Petri dishes. After cooling to room temperature, holes were punched into the solidified agar. 50 μL of undiluted eluate or 50 μL of eluate diluted 1:50 were added to the punched holes in the nutrient agar. Aqueous gentamicin sulfate solution, vancomycin hydrochloride solution, and clindamycin hydrochloride solution were also included as controls. The agar plates were then incubated for 24 hours at 37° C. FIGS. 2 and 3 each show a schematic distribution of the different samples (labeled HE-3 and HE-4) on the plate, and also an actual image of the plate at the end of the experiment.

TABLE 14
Description of the different samples applied
to the agar plate; corresponding to FIG. 2.

| Plate | Punched | | Dilution |
|---|---|---|---|
| name | hole | Sample | with water |
| HE-3 | 1 | Reference solution gentamicin sulfate | — |
| | | (concentration 20 μg/mL gentamicin | |
| | | base) | |
| | 2 | Reference solution gentamicin sulfate | — |
| | | (concentration 10 μg/mL gentamicin | |
| | | base) | |
| | 3a | Reference solution vancomycin | — |
| | | hydrochloride (concentration 5 μg/mL | |
| | | vancomycin base) | |
| | 4 | Mixture 4 | 1:50 |
| | 5 | Mixture 8 | 1:50 |
| | 6 | Mixture 6 | 1:50 |
| | 3b | Reference solution clindamycin | — |
| | | hydrochloride (concentration 2 μg/mL | |
| | | clindamycin base) | |

TABLE 15
Description of the different samples applied
to the agar plate; corresponding to FIG. 3.

| Plate | Punched | | |
|---|---|---|---|
| name | hole | Sample | Dilution |
| HE-4 | 1 | Reference solution gentamicin sulfate | — |
| | | (concentration 20 μg/mL gentamicin base) | |
| | 2 | Reference solution gentamicin sulfate | — |
| | | (concentration 10 μg/mL gentamicin base) | |
| | 3a | Reference solution vancomycin | — |
| | | hydrochloride (concentration 5 μg/mL | |
| | | vancomycin base) | |
| | 4 | Mixture 10 | 1:50 |
| | 5 | Mixture 8 | 1:50 |
| | 6a | Reference solution clindamycin | 1:50 |
| | | hydrochloride (concentration 4 μg/mL | |
| | | clindamycin base) | |
| | 3b | Reference solution clindamycin | — |
| | | hydrochloride (concentration 2 μg/mL | |
| | | clindamycin base) | |
| | 6b | Reference solution vancomycin | |
| | | hydrochloride (concentration 10 μg/mL | |
| | | vancomycin base) | |

### DESCRIPTION OF THE FIGURES

FIG. 1 Scanning electron micrographs of a coating produced according to the invention a) before incubation of the carrier for 24 h at 37° C. and b) images of the same coating after the incubation process. a) shows a coating in which inclusions of active ingredient and possibly unpolymerized material in the form of closed bubbles can be seen. b) shows the same coating, but the inclusions have opened, which can be seen by the dark-colored openings.

FIG. 2 Agar plate inoculated with bacteria HE-3, schematic sample distribution a) and image of the carrier after incubation for 24 h at 37° C. b); The sample descriptions shown in Table 14 correspond to the scheme shown under a). In each case, one of the sample solutions shown in Table 14 has been applied to one of the punched holes visible as dark circles in b) and then diffused circularly into the bacterial culture. The inhibition zones in which the bacterial concentration has decreased are visible as circular areas around the cavities.

FIG. 3 Agar plate inoculated with bacteria HE-4, schematic sample distribution a) and image of the carrier after incubation for 24 h at 37° C. b); The sample descriptions shown in Table 15 correspond to the scheme shown under a). In each case, one of the sample solutions shown in Table 15 has been applied to one of the punched holes visible as dark circles in b) and then diffused circularly into the bacterial culture. The inhibition zones in which the bacterial concentration has decreased are visible as circular areas around the cavities.

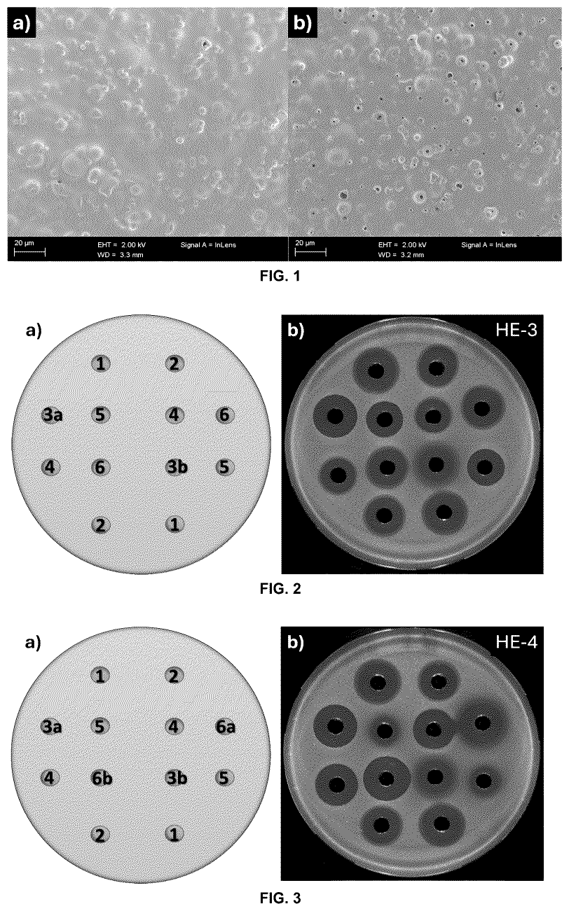

The invention claimed is:

1. A method for producing a pharmaceutically active polymer coating, comprising the following method steps:
a) dissolving a non-hydrolyzable polymer or copolymer which is characterized by a number-average molar mass of greater than 200,000 g/mol, more preferably a number-average molar mass of greater than 400,000 g/mol, more preferably a number-average molar mass of greater than 600,000 g/mol, determined by gel permeation chromatography, in a solvent or solvent mixture which is characterized by a vapor pressure of at least 95 hPa at 20° C. and standard pressure, forming a polymer solution 1;
b) dissolving or suspending at least one pharmaceutical active ingredient in the polymer solution 1, forming a polymer solution 2;
c) applying the polymer solution 2 to the surface of an article to be coated; and
d) evaporating the solvent or solvent mixture so that a polymer layer remains on the surface of the article to be coated, in which polymer layer the at least one pharmaceutical active ingredient is dispersed,
wherein the polymer layer does not comprise any pore-forming agents which are soluble in aqueous solutions.

2. The method according to claim 1, wherein the polymers or copolymers have a glass transition temperature of greater than 47° C., more preferably greater than 60° C., more preferably greater than 80° C., more preferably greater than 100° C.

3. The method according to claim 1, wherein the polymer or copolymer is based upon polymethyl methacrylates and, in the case of copolymers, the comonomer is selected from the group consisting of methyl acrylate, ethyl acrylate, ethyl methacrylate, styrene, and mixtures thereof.

4. The method according to claim 1, wherein the solvent or solvent mixture has a water content of less than 8 wt. %, more preferably less than 5 wt. %, more preferably less than 2 wt. %.

5. The method according to claim 1, characterized in that the solvent or solvent mixture is selected from the group consisting of acetic acid esters, acetoacetic acid esters, acetone, diacetone alcohol, ethyl methyl ketone, butyl methyl ketone, and mixtures thereof.

6. The method according to claim 1, wherein the polymer or copolymer content in the polymer solution 1 is between 2 wt. % and 18 wt. %, preferably between 9 wt. % and 11 wt. %.

7. The method according to claim 1, wherein the pharmaceutical active ingredient is selected from the group consisting of anti-infectives, bacteriophages, cytostatics, analgesics, hormones, growth factors, and mixtures thereof.

8. The method according to claim 1, wherein the pharmaceutical active ingredient in step b) is added as a particulate solid having a particle size of less than 250 μm, more preferably having a particle size of less than 100 μm, determined by means of sieve fractionation, for being dissolved or suspended in the polymer solution 1 to obtain a polymer solution 2.

9. The method according to claim 1, wherein the weight ratio of polymers or copolymers to pharmaceutical active ingredients is 1.00:0.05 to 1.00:1.50.

10. The method according to claim 1, wherein the polymer layer has a layer thickness of 50 to 500 μm.

11. A medical kit, in particular for carrying out the method according to claim 1, comprising the following components:
i) a solvent-resistant container having an opening;
ii) a solvent-resistant closure element suitable for closing the container;
iii) an application aid;
iv) a polymer solution 1 obtained according to the method according to one of claims 1 to 10;
v) optionally, one or more mixing bodies; and
vi) optionally, one or more pharmaceutical active ingredients in separate packaging.

12. The medical kit according to claim 11, wherein
the opening of the container has a diameter of at least 1 cm, preferably at least 2 cm, more preferably at least 3 cm, and/or
application aids are selected from brushes or atomizer caps, and/or
the mixing body is selected from at least one mixing rod and/or at least one mixing element provided in the container in the polymer solution 1, and/or
in that one or more of the elements listed under components i) to iii) of the medical kit are attached to one another.

13. A device for carrying out the method for producing a pharmaceutically active polymer coating, comprising the components of the medical kit according to claim 11.

14. A medical implant coated with a polymer coating having a dispersed pharmaceutical active ingredient, obtained by a method according to claim 1.

15. The medical implant according to claim 14, for preventing infections after a surgical procedure.
